(19) United States
(12) Patent Application Publication
(10) Pub. No.: US 2017/0166909 A1
(43) Pub. Date: Jun. 15, 2017

(54) IMPROVED STRAINS OF AGROBACTERIUM TUMEFACIENS FOR TRANSFERRING DNA INTO PLANTS

(71) Applicant: RHEINISCHE FRIEDRICH-WILHELMS-UNIVERSITÄT BONN, BONN (DE)

(72) Inventors: BEKIR ÜLKER, BONN (DE); TOBIAS BERSON, BONN (DE)

(21) Appl. No.: 15/328,020

(22) PCT Filed: Jul. 21, 2015
(86) PCT No.: PCT/EP2015/066667

§ 371 Date: Jan. 21, 2017

(30) Foreign Application Priority Data
Jul. 23, 2014 (EP) 14002560.2

(51) Int. Cl. C12N 15/74; C12N 15/82

(52) U.S. Cl. CPC C12N 15/743; C12N 15/8205

(57) ABSTRACT

The present invention relates to Agrobacterium tumefaciens strains that comprise at least one deletion/mutation in a sequence selected from the group of IS426 copy I, IS426 copy II, the OriT-like sequence, and the border-like sequences, and their uses in safer and improved transformation procedures for cells.

The present invention relates to Agrobacterium tumefaciens strains that comprise at least one deletion/mutation in a sequence selected from the group of IS426 copy I, IS426 copy II, the OriT-like sequence, and border-like sequences, and their uses in safer and improved transformation procedures for cells.

### BACKGROUND OF THE INVENTION

Agrobacterium tumefaciens is the workhorse of plant molecular biology and plant genetic engineering as this bacterium can efficiently transform plants. Methods using the bacterium have also been successfully used in transforming numerous other organisms, including human cells. However, as was demonstrated in 2008, the available Agrobacterium strains have hidden biosecurity risks.

In addition to the DNA of interest (i.e. to be transformed) contained within the T-DNA, sometimes very large other fragments of bacterial chromosomal DNA (AchrDNA) are also unintentionally transferred from the bacteria into plants (Ulker et al., 2008). Thus, besides the well-documented integration of DNA flanked by the transfer DNA borders, occasional insertion of fragments from the tumor-inducing plasmid into plant genomes has also been reported during Agrobacterium tumefaciens—mediated transformation. Large (up to 18 kb) gene-bearing fragments of Agrobacterium chromosomal DNA (AchrDNA) can be integrated into, for example, Arabidopsis thaliana genomic DNA during transformation. About one in every 250 transgenic plants may carry AchrDNA fragments. This has implications for horizontal gene transfer and indicates a need for greater scrutiny of transgenic plants for undesired bacterial DNA, as Agrobacterium tumefaciens still is a soil-borne bacterial pathogen of plants.

In nature, Agrobacterium transfers a defined segment of the tumor inducing (Ti) plasmid (T-DNA) into the host, leading to the formation of crown gall tumors controlled by T-DNA-encoded oncogenes. Agrobacterium-mediated DNA transfer has been exploited to introduce transgenes into plants and to transform other organisms such as yeast, fungi and even human cells. Sometimes, part of the Ti plasmid outside the T-DNA borders may be integrated into plant genomes. The A. tumefaciens strain C58 genome of 5.7 megabases has been completely sequenced and comprises four replicons: a linear chromosome, a circular chromosome and the two large plasmids AtC58 and TiC58.

Until the experiments that led to the present patent application, the mechanisms of how these chromosomal DNAs are transferred from bacteria to plants were not known. The present inventors have now determined the key mechanisms involved in this process.

It is therefore an object of the present invention to provide biosafe Agrobacterium strains that are less prone to the undesired transfer of DNA as described above. Furthermore, methods for improved transfer of DNA shall be developed. Other objects and advantages of the present invention will become apparent to the person of skill upon studying the following description of the present invention.

According to a first aspect of the present invention, the object is solved by providing an Agrobacterium tumefaciens strain, comprising at least one deletion and or mutation functionally inactivating said sequence in a sequence selected from the group of IS426 copy I, IS426 copy II, the OriT-like sequence, and the border-like sequences, for example the left- (LB) or right-border (RB)-like sequences. Preferred is at least one deletion.

The sequences to be mutated or deleted can also be selected from sequences with a nucleotide sequence that is at least 80%, more preferably, 90%, even more preferably 95% or 98% or 99% identical to the sequence of IS426 copy I, IS426 copy II, the OriT-like sequence, or the border-like sequences, for example the left- (LB) or right-border (RB)-like sequences, such as, for example, a sequence according to SEQ ID No. 1, 2, 3, 7, 8, 9 or 10.

According to the present invention, the term “IS426” relates to the sequence as disclosed in SEQ ID No. 1, or according to GenBank Accession No. X56562.1. The other sequences relate to the sequence of plasmid TiC58 (Accession No. NC_003065.3) and as disclosed in Ulker, B. et al. (Nat Biotechnol 26, 1015-1017).

Preferred is the Agrobacterium tumefaciens strain according to the present invention, wherein said strain comprises said deletion and/or inactivating mutation in two, three, or all four of said sequences.

An inactivating mutation in the context of the present invention shall mean a mutation that, when introduced into the elements as described herein, reduces, substantially reduces, or even abolishes the undesired transfer of DNA as described herein. Such mutation can be selected from a point mutation, but also includes several point mutations and/or added nucleotides for inactivation.

Vanderleyden J, et al. (in: Nucleotide sequence of an insertion sequence (IS) element identified in the T-DNA region of a spontaneous variant of the Ti-plasmid pTiT37. Nucleic Acids Res. 1986 Aug. 26; 14(16): 6699-709) describe the nucleotide sequence of an IS element (IS136, synonym of 1S426) of Agrobacterium tumefaciens. The IS element has 32/30 bp inverted repeats with 6 mismatches, is 1,313 bp long and generates 9 bp direct repeats upon integration. IS136 has 3 main open reading frames (ORF's). Only ORF1 (159 codons) is preceded by sequences that are proposed to serve functional roles in transcriptional and translational initiation. No DNA sequence homology was found between IS136 and IS66, an IS element isolated from an octopine type Ti-plasmid.

Further preferred is the Agrobacterium tumefaciens strain according to the present invention, wherein said deletion of said sequence is partially or fully, such as, for example, 30 bp in the RB-like sequence, and/or 61 bp in the OriT-like sequence.

Further preferred is the Agrobacterium tumefaciens strain according to the present invention, wherein the OriT-like sequence is located in the HS1_{LC} region and the RB-like sequence is located in the HS1_{CC} region.

As mentioned above, Agrobacterium tumefaciens is the workhorse of plant molecular biology and genetic engineering, as this bacterium can efficiently transform plants, which gave rise to the Agrobacterium-mediated transformation methods that have been the methods of choice when transforming plants. Numerous commercial transgenic crops generated using this technology are cultivated in several countries and are used in food, feeding or other industries. The methods have also been successfully used in transforming other organisms including human cells. Agrobacterium is a paradigm model for TypeIV SS employed by many human pathogenes such as Helicobacter, Bartonella and Legionella. Agrobacterium-mediated plant transformation is also the method of choice in most cases because it is a simple procedure, produces a high transformation efficiency, most plant species can be transformed, requires only a low transgene copy number, and can be done in basically every laboratory (S1).

While characterizing a T-DNA insertion locus named PM within the fully sequenced A. thaliana genome, the inventors discovered a 322-bp DNA fragment of non-plant origin associated with the right border (RB) of the T-DNA. The finding that this sequence is identical to a region on the sequenced linear chromosome of A. tumefaciens led the inventors to determine, whether this was a unique event or whether it is an intrinsic property associated with T-DNA transfer in general. Therefore, the inventors analyzed databases that contain A. thaliana-flanking sequence tags (FSTs), the sequences that flank T-DNA insertion sites in populations of insertion lines generated to saturate the genome with mutations. Fragments of AchrDNAs were detected in all tested T-DNA insertion databases, and AchrDNAs were found much more frequently in FSTs recovered from the RB. Based on these data, obtained from >375,000 T-DNA-tagged A. thaliana lines, the inventors estimated that about 0.4% (from the RB FSTs of GABI-Kat) of the insertion sites actually contain bacterial chromosomal DNA. The different populations as studied had been generated with different T-DNA vectors and A. tumefaciens strains, indicating that fragments of AchrDNA are transferred to the plant genome irrespective of the binary vector or A. tumefaciens strain used. In addition, the inventors also studied rice FST collections and detected AchrDNA sequences, indicating that the transfer of AchrDNAs through Agrobacterium happens in rice as well (Üliker et al., 2008).

The present invention is based on the surprising finding that several genetic elements can be held responsible for the vast majority of the undesired transfer events. These are short DNA regions (cis-elements) in Agrobacterium chromosomes which are responsible for transfer of flanking bacterial DNAs to plant genomes during plant transformation. Identified were a) the OriT-like (origin of transfer like) region found in the HS1_{LC} (hot spot 1 on Agrobacterium linear chromosome) which is responsible for the majority of the AchrDNA transfer from the linear Agrobacterium chromosome, and b) the RB-like (right border like) element found on the HS1_{CC} (hot spot 1 on Agrobacterium circular chromosome) and is responsible transferring an AchrDNA region in the circular chromosome. Thus, the presence of at least one of these elements constitutes a biosafety risk. Furthermore, IS426, a particularly active insertion sequence (transposon) which has two full length copies (IS426 copy I and IS426 copy II), one partial and circular transposition intermediate copy, has been identified. The transposon can jump into the T-DNA regions in plant transformation vectors and with T-DNA is transferred to plants. As was furthermore shown, IS426 can also mutate or activate genes (especially antibiotic resistance genes) in bacteria. Therefore, IS426 is also a particular biosafety risk factor.

The present inventors now showed that the problematic genetic elements as identified can be removed (e.g. deleted) from Agrobacterium genome without a negative effect on the viability or effectiveness of the bacteria, as the deletion thereof does not influence normal and desired T-DNA transformation processes. The strains as produced still contain the remnants of antibiotic resistance cassettes as introduced to aid selection of homologous recombination mediated deletion events. Nevertheless, such antibiotic resistance genes can readily be removed from the strain by the person of skill in order not to limit the number of selectable marker genes that can be used when engineering this bacterium further. The following strains were constructed and tested.

The desired deletions can be introduced into the Agrobacterium tumefaciens strains according to the invention by using any method known to the person of skill, such as, for example, using suicide vectors comprising suitable resistance markers, such as, for example, antibiotic resistance markers, such as kanamycin resistance.
An IS426 copy I deletion strain in an Agrobacterium tumefaciens A136 background;
An IS426 copy II deletion strain, also in an Agrobacterium tumefaciens A136 background;
An IS426 copy I and IS426 copy II deletion strain in an Agrobacterium tumefaciens A136 background; and
An OriT deletion strains in an Agrobacterium tumefaciens GV3101 pMP90 background.

Encompassed by the present invention is an RB-like element deletion strain, which can be generated in Agrobacterium tumefaciens in analogy to the above strains. Further included are an IS426 copy I, IS426 copy II deletion, and OriT deletion strain in an Agrobacterium tumefaciens background; and an IS426 copy I and OriT deletion strain in an Agrobacterium tumefaciens background; and an IS426 copy II deletion and OriT deletion strain in an Agrobacterium tumefaciens background.

In a particularly preferred embodiment of the present invention, the invention relates to an IS426 copy I, IS426 copy II deletion, RB-like element and OriT element deletion strain in an Agrobacterium tumefaciens background. It is expected that this strain will be nearly devoid of transforming or transferring undesired sequences into the cell to be transformed. The term “deletion strain” also encompasses strains carrying functionally inactivating mutations.

In a particularly preferred embodiment of the present invention, a desired bacterium has the genotype of deletions of two full length and active insertion sequences, IS426 copy I and IS426 copy II from the linear chromosome, a deletion of 61 bp OriT-like in the HS1_{LC} region on the linear chromosome, and, a deletion of 30 bp RB-like sequence in the HS1_{CC} on the circular chromosome, as described also below.

In yet another aspect of the present invention, the invention relates to an Agrobacterium tumefaciens strain according to the present invention, further comprising a recombinant chromosomally integrated minimal TypeIV secretion system (TypeIV SS), optionally comprising virD2. This will simplify plant transformation because there will be no more need for the use of a binary vector system, and thus helper plasmids. The bacteria will grow faster, and the use of antibiotic resistance genes is minimal, which will allow a better use of the markers in molecular biology work involving Agrobacterium as a host.

In yet another aspect of the present invention, the invention then relates to a method for producing an Agrobacterium tumefaciens strain according to the present invention, comprising the step of introducing at least one deletion in a sequence selected from the group of IS426 copy I, IS426 copy II, the OriT-like sequence, and the RB-like sequence in said strain.

Preferred is a method for producing an Agrobacterium tumefaciens strain, additionally comprising introducing a recombinant chromosomally integrated minimal TypeIV secretion system (TypeIV SS), optionally comprising virD2, into an Agrobacterium tumefaciens strain according to the present invention.

In yet another aspect of the present invention, the invention then relates to a method for transforming a cell selected from the group consisting of a plant, yeast, fungal, and human cell with a recombinant nucleic acid, comprising contacting said cell with an Agrobacterium tumefaciens strain according to the present invention, wherein said strain carries said recombinant nucleic acid to be transformed. Respective methods for Agrobacterium tumefaciens transformation are very well known in the art, and can be readily adapted by the person of skill. The present Agrobacterium tumefaciens strains do not require substantially different transformation conditions, compared to a non-modified Agrobacterium tumefaciens strain.

The present invention provides a number of important improvements for the field of cellular (In particular plant) transformation using the Agrobacterium T-DNA transformation system (also known as Agrobacterium-mediated (plant) transformation). The system and the strains of the present invention can be used to generate transgenic crops, to analyze the role of chromosomal DNA transfer in bacteria host interactions and disease development, for the transformation of yeast and fungi, and even for the transformation of animal and human cells. The system can be used to deliver proteins/genes designed or produced in Agrobacterium into these cells as well.

The present invention will now be further explained in the following examples with reference to the accompanying figures, nevertheless, without being limited thereto. For the purposes of the present invention all references as cited herein are incorporated by reference in their entireties. In the Figures,

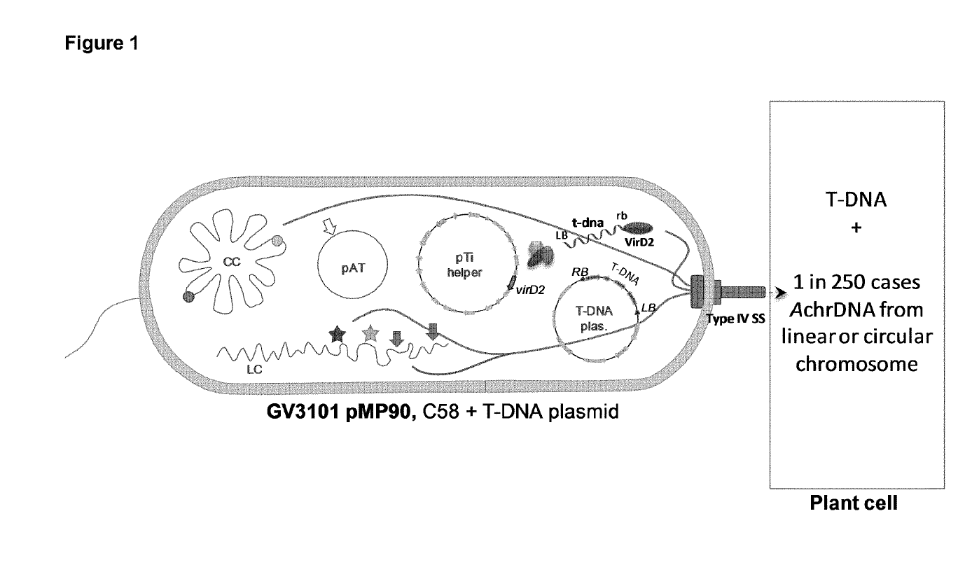

FIG. 1 shows that in addition to the T-DNA, Agrobacterium transfers very large fragments of its chromosomal DNA (AchrDNA) into plants.

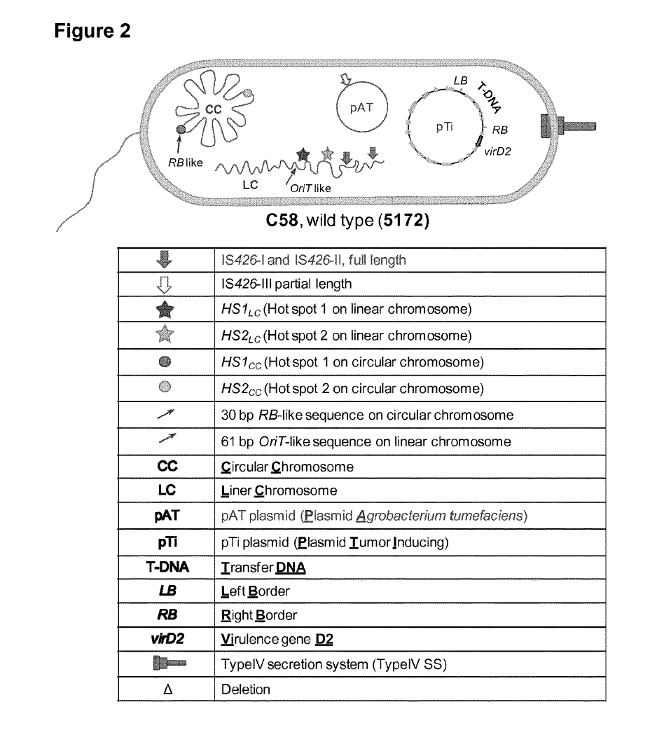

FIG. 2 shows acronyms and labels used to describe genotypes of Agrobacterium strains as generated and used in this patent application.

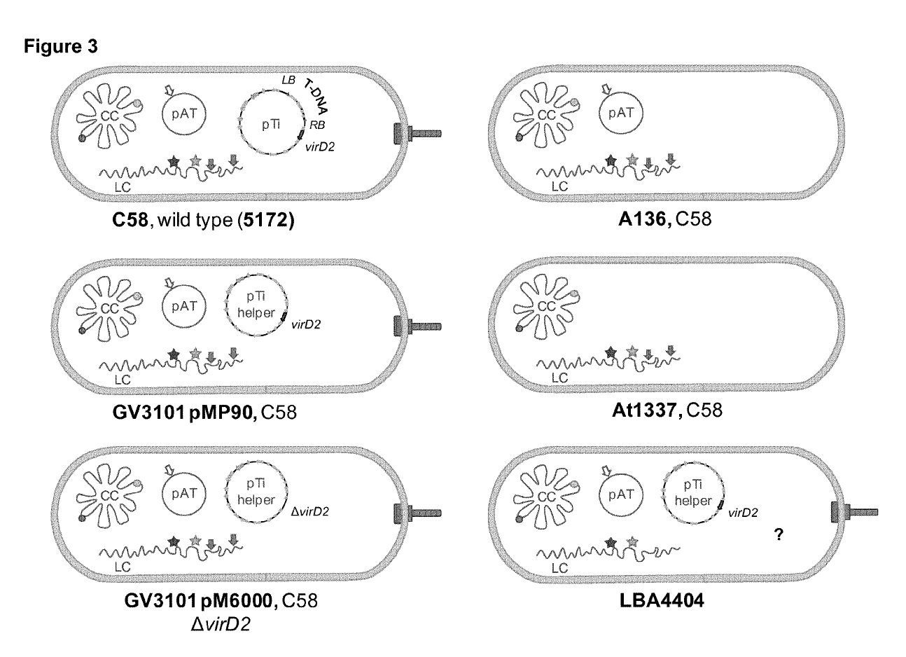

FIG. 3 shows the strains used to determine mechanisms of bacterial chromosomal DNA transfer.

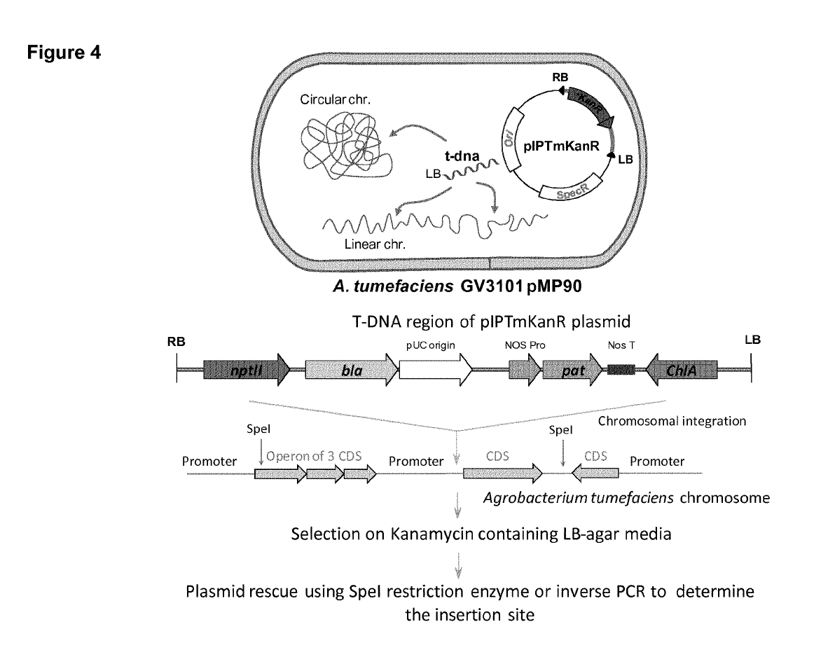

FIG. 4 shows the promoter trapping assay used in the determination of bacterial chromosomal DNA transfer.

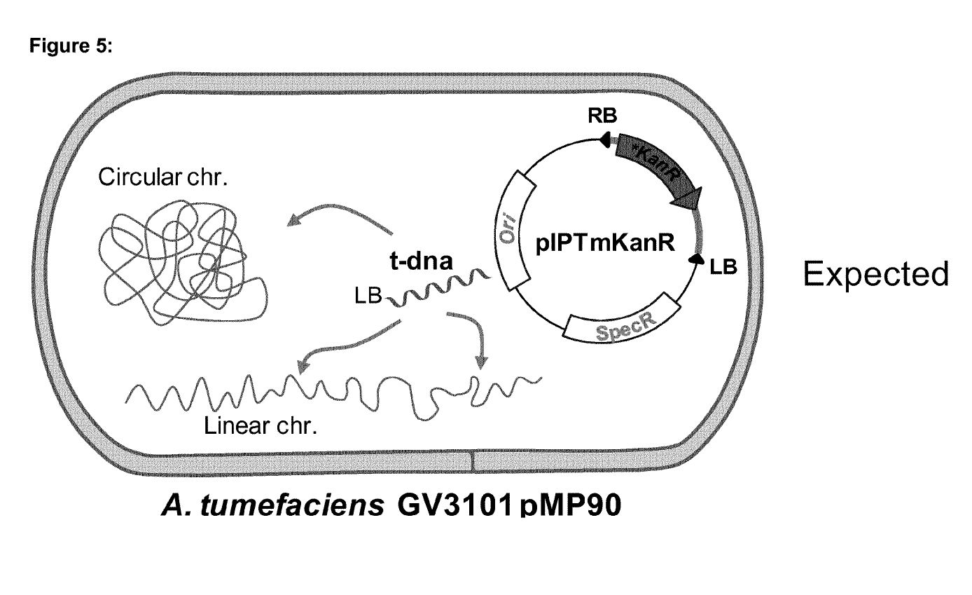

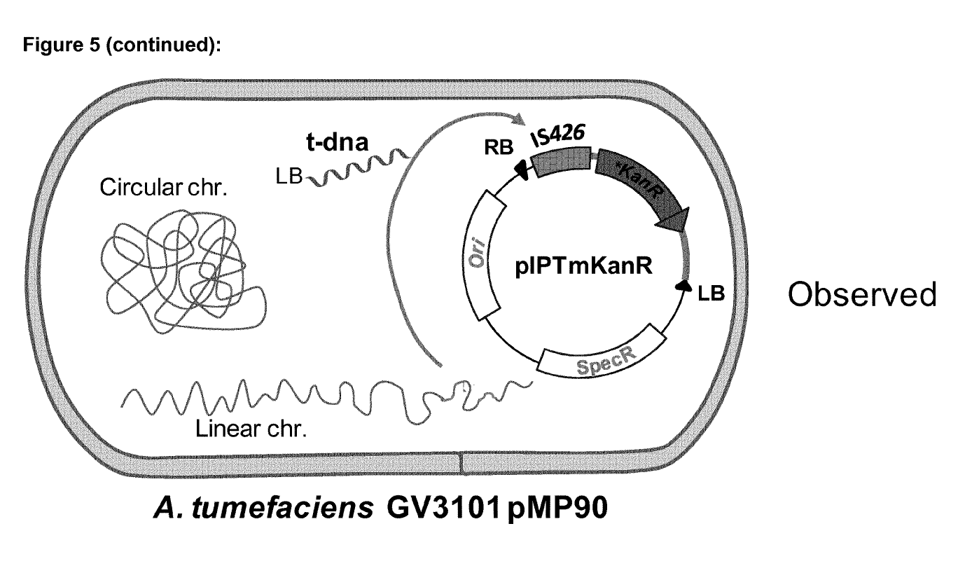

FIG. 5 schematically shows an active insertion sequence in Agrobacterium tumefaciens genome that was identified using the promoter trapping assay.

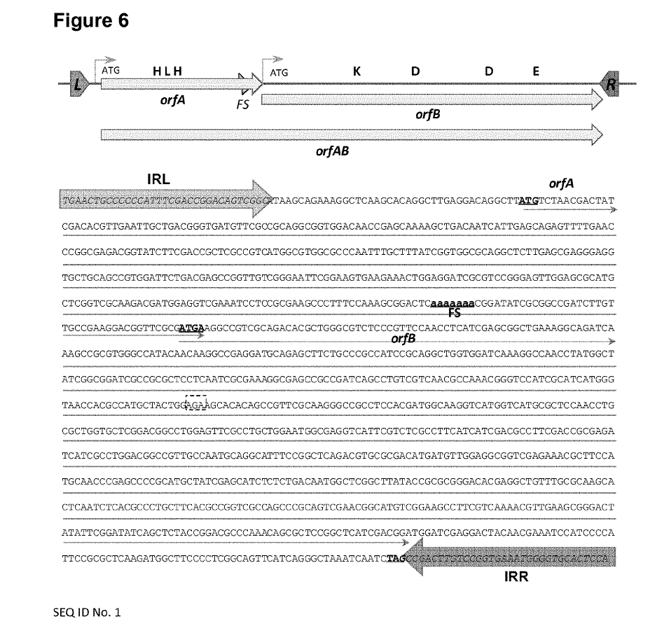

FIG. 6 shows a schematic depiction of the structure and putative coding sequences within IS426.

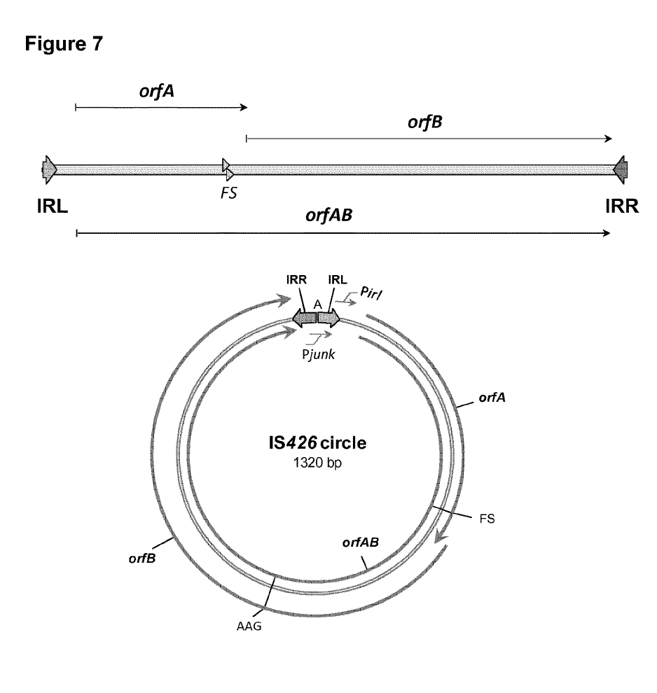

FIG. 7 shows a schematic depiction of the PCR analysis and sequencing of the PCR products that led to the detection of IS426 circles (possible transposition intermediates).

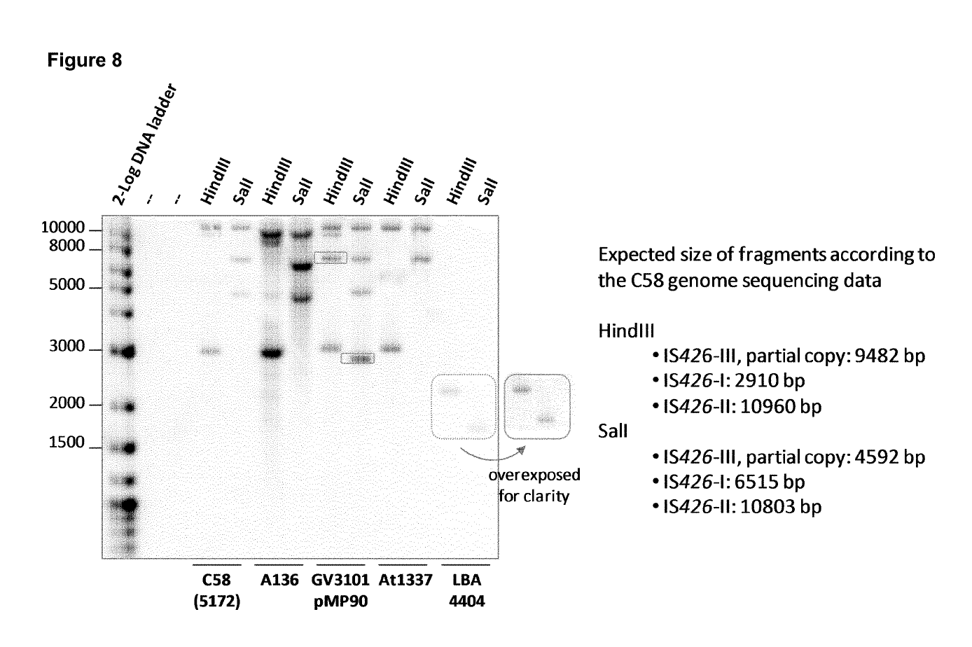

FIG. 8 shows the results of the southern blot analysis to determine IS426 copy numbers in selected Agrobacterium strains.

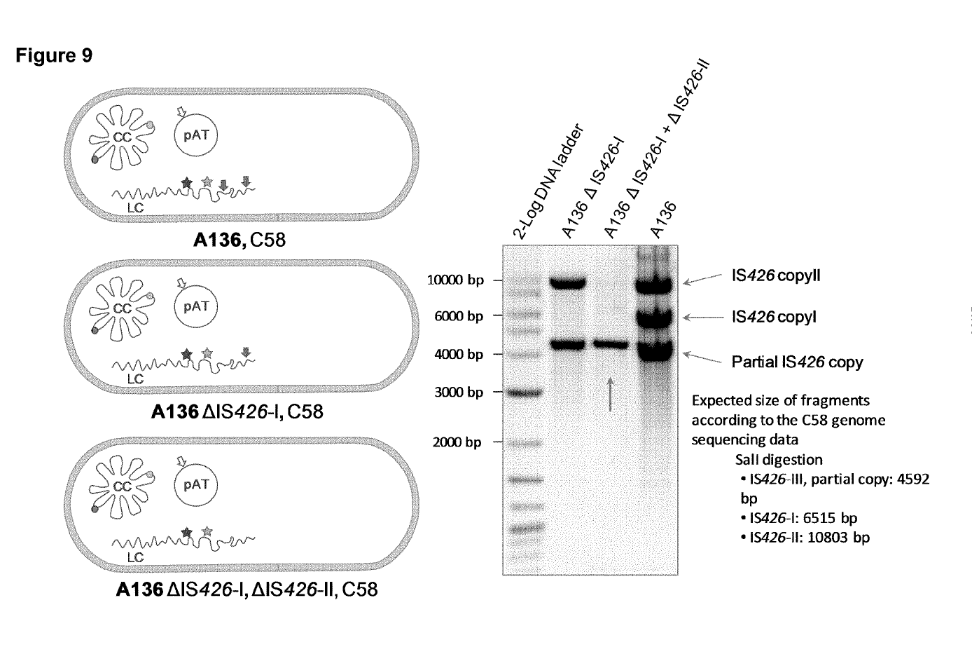

FIG. 9 shows the generation of IS426 deletion strains of Agrobacterium. The Southern blot analysis to determine IS426 copy numbers in engineered, non-virulent, cured Agrobacterium strain A136 is shown (right side) in order to indicate indeed that both full copies are deleted.

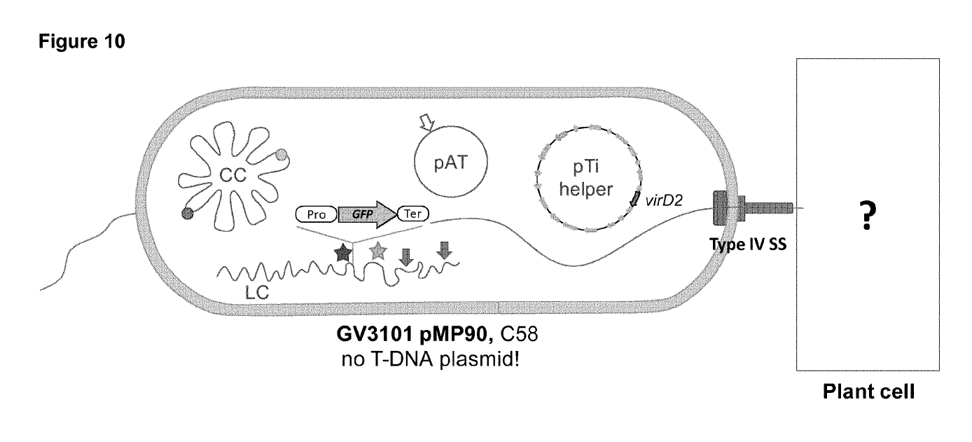

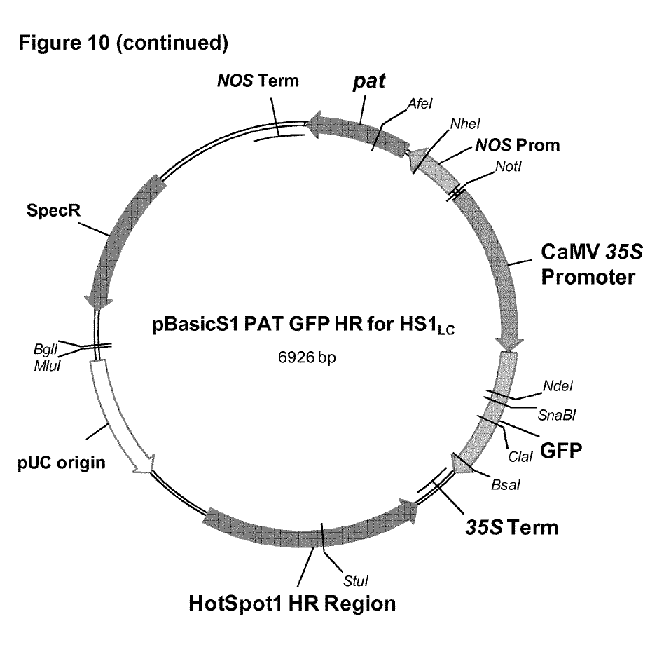

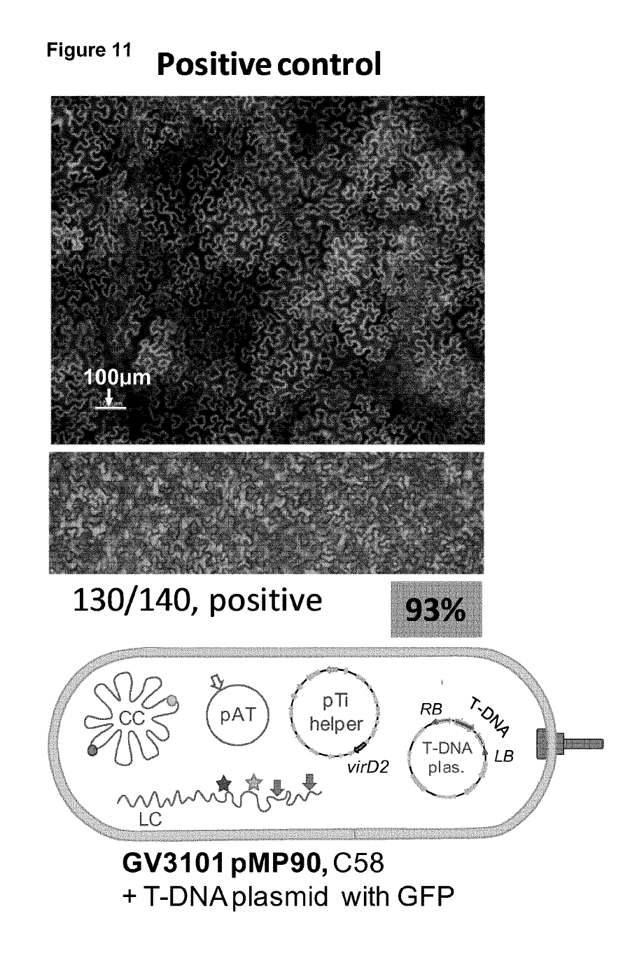

FIG. 10 shows a schematic depiction of Agrobacterium chromosomal DNA hot spots that were labelled by inserting a GFP expression cassette (active only in plants).

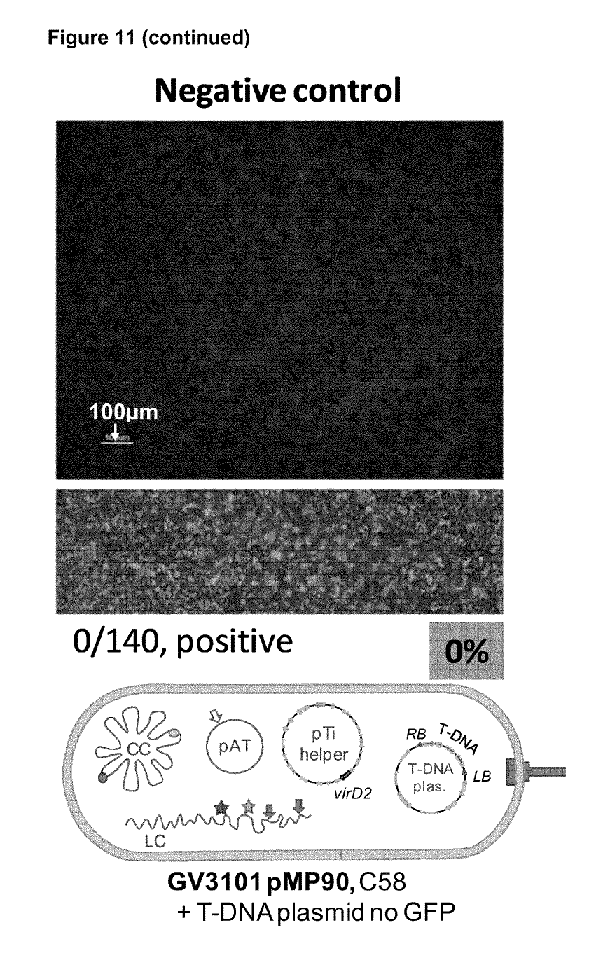

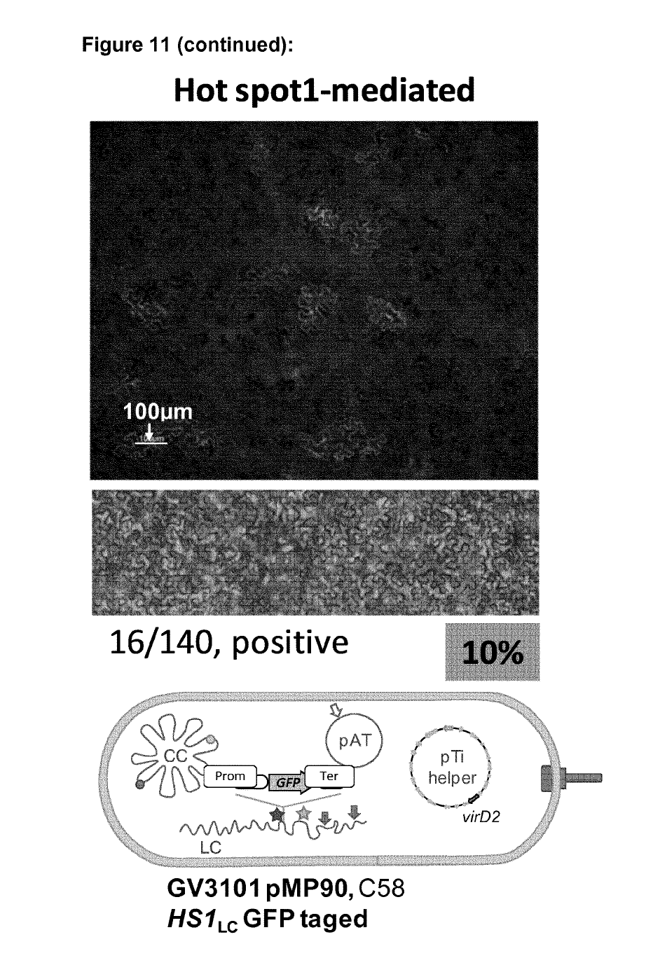

FIG. 11 shows that HS1_{LC} tagged with GFP shows that Agrobacteria transfer the tagged region into plants.

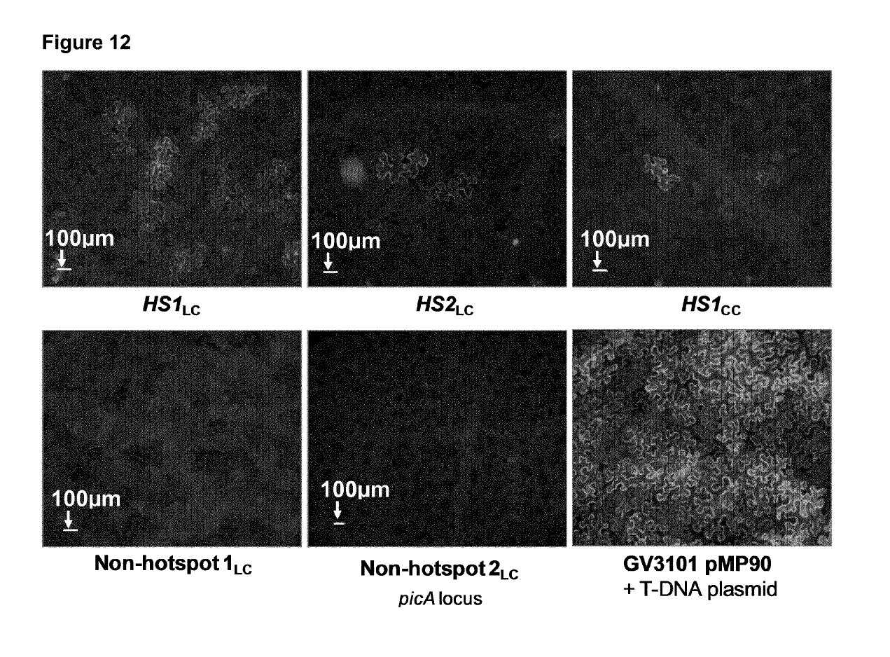

FIG. 12 shows the results of tagging other hot spots and non-hot spots with GFP in GV3101 pMP90 Agrobacterium strain and plant transformation using the transient assay.

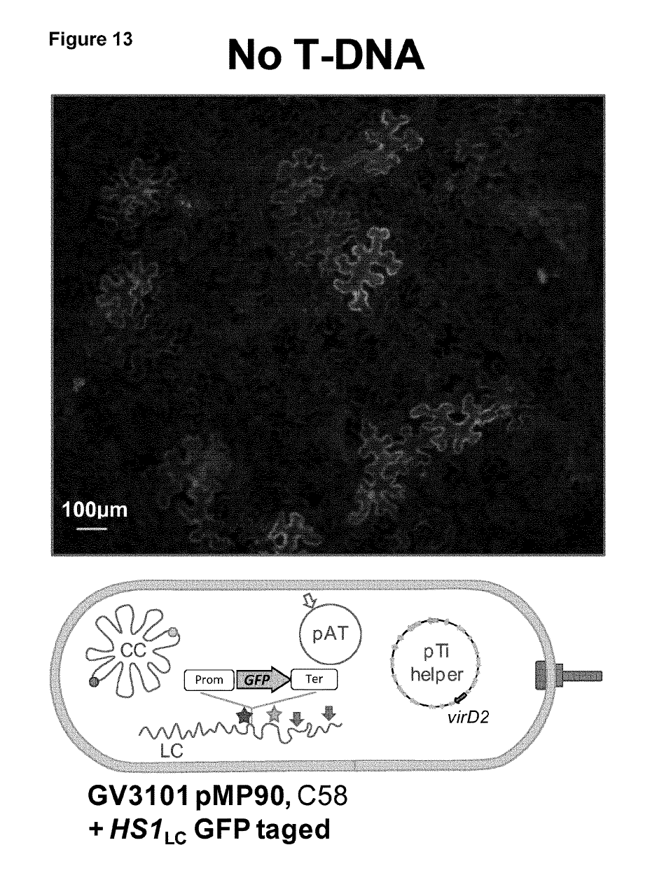

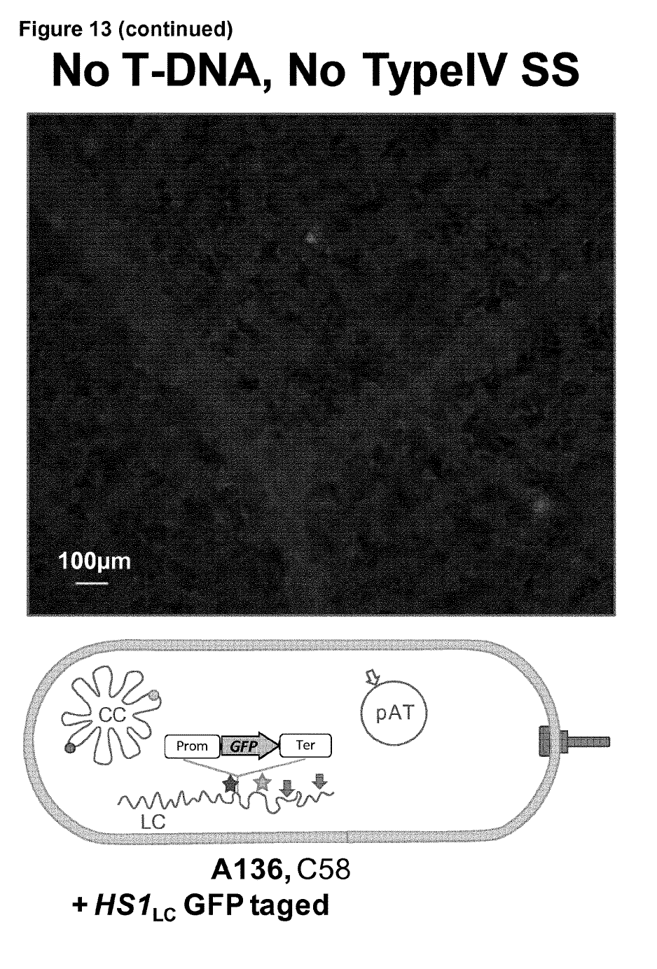

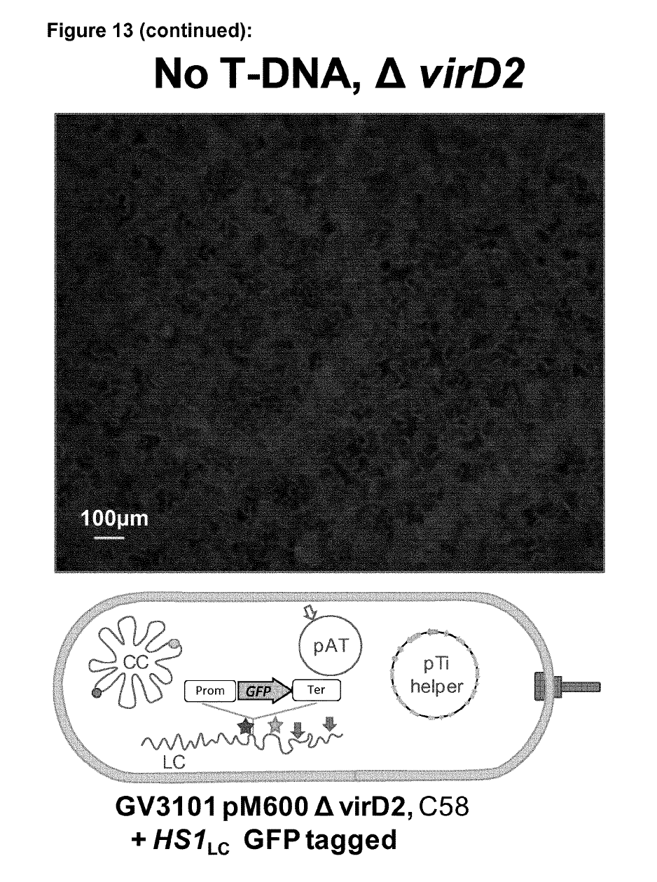

FIG. 13 shows that tagging HS1_{LC} with GFP in selected Agrobacterium strains identified that chromosomal DNA is VirD2 and TypeIV SS dependent.

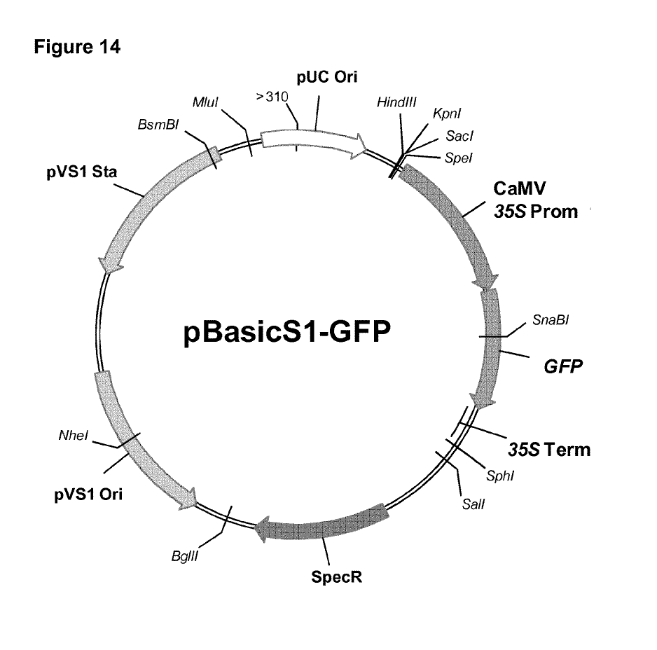

FIG. 14 shows the map of pBasicS1-GFP vector used in testing cis elements involved in chromosomal DNA transfer.

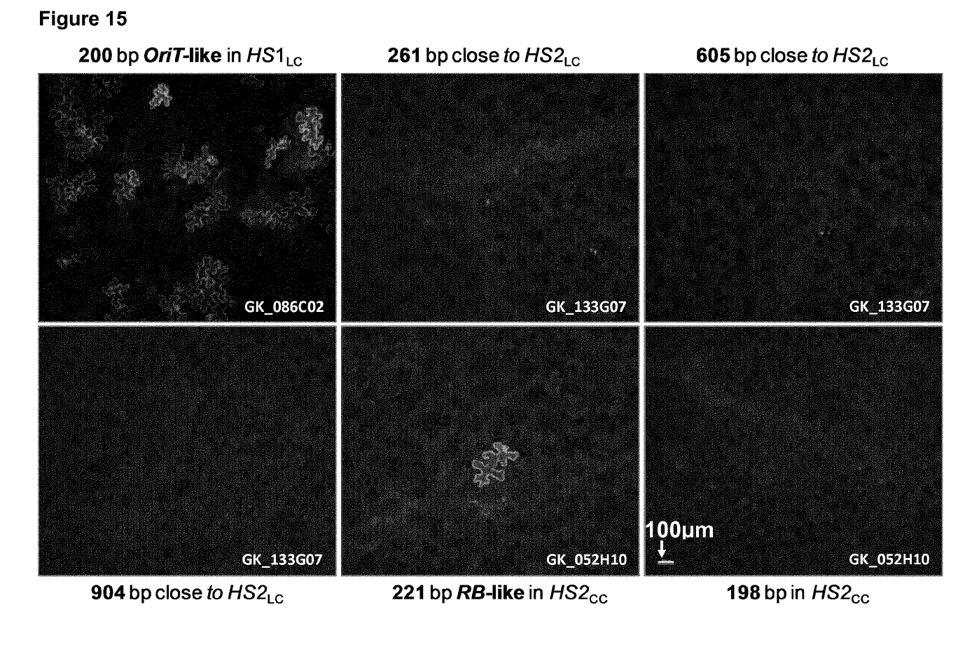

FIG. 15 shows the results of the searches for DNA regions at or around the hot spots that led to the discovery of OriT-like and RB-like sequences responsible for AchrDNA transfer.

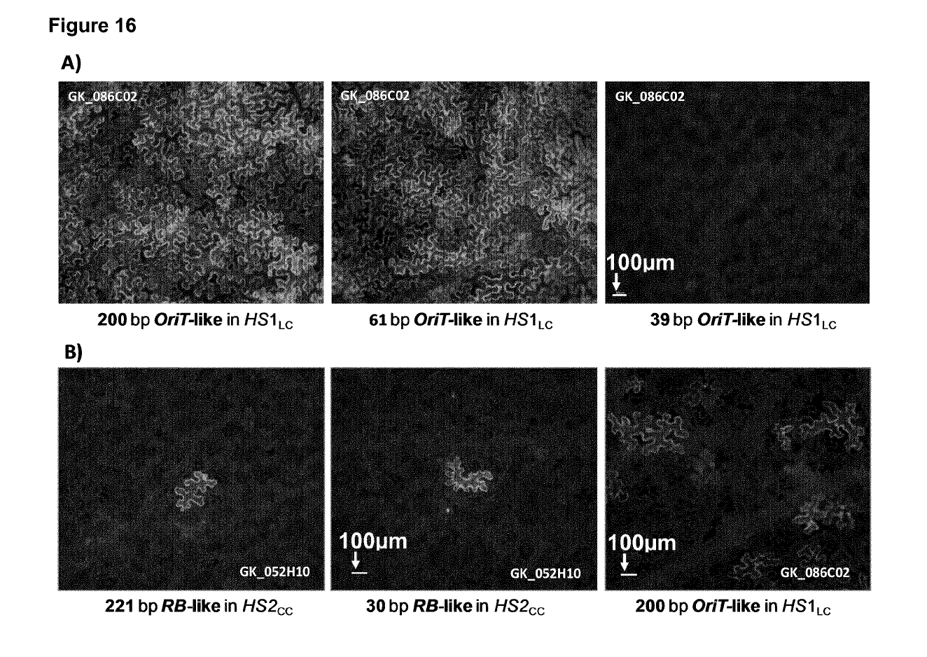

FIG. 16 shows that A) the predicted OriT-like sequence in the linear chromosome hot spot 1 is responsible for transferring this region from bacteria to plants, and B) that the predicted RB-like sequence in the circular chromosome hot spot 1 is responsible for transferring this region from bacteria to plants.

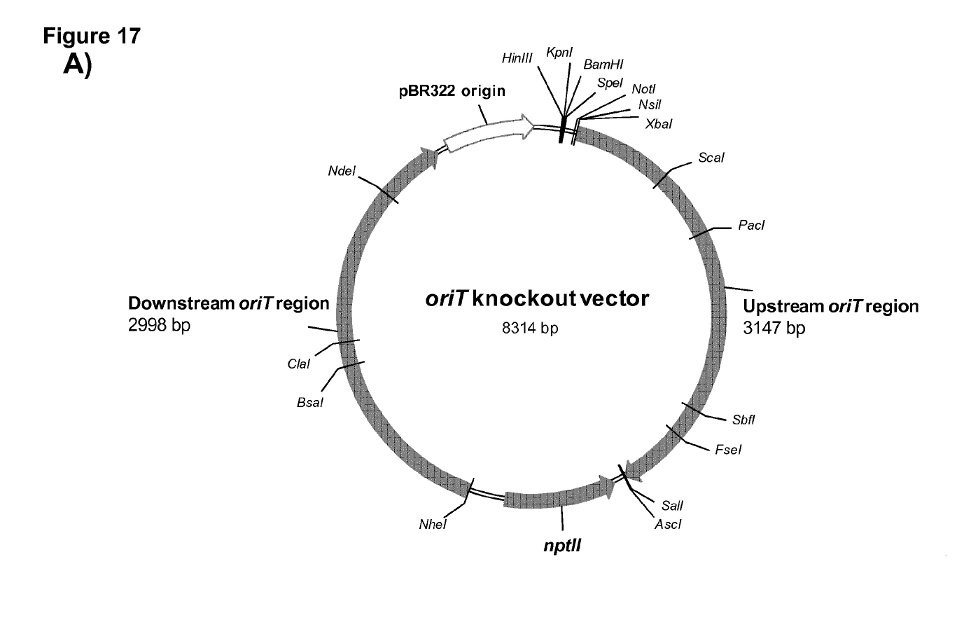

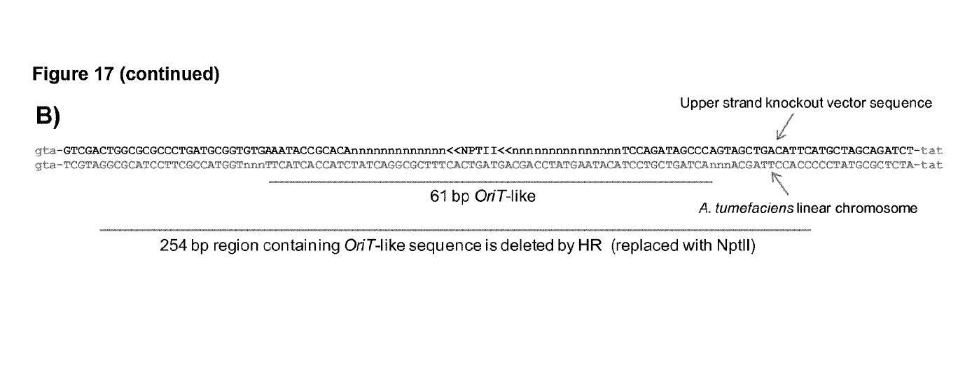

FIG. 17 shows the strategy used in deletion of the OriT-like sequences is depicted. (A) The recombination vector is a suicide plasmid and cannot replicate in Agrobacterium. It contains bacterial expression cassette for the kanamycin resistance gene nptII flanked by the Agrobacterium sequences determining the position of recombination mediated deletion of sequences from bacterial genome but addition of nptII expression cassette. (B) Transformation of the recombination vector into HS1_{LC} GFP-tagged GV3101 pMP90 Agrobacterium cells and selection of bacteria by kanamycin resulted in double recombination mediated replacement of nptII with the OriT-like sequence on the linear chromosome

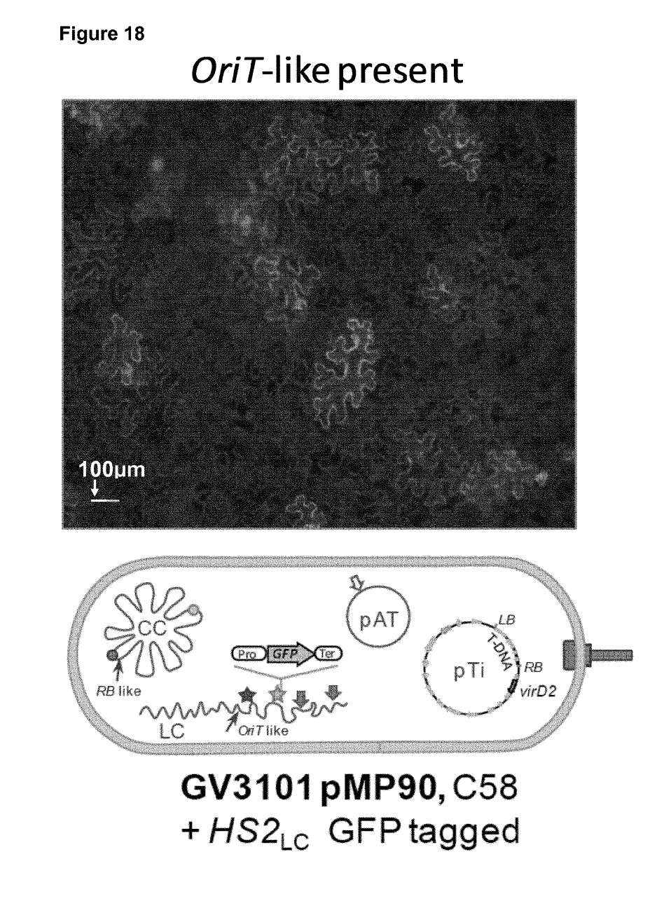

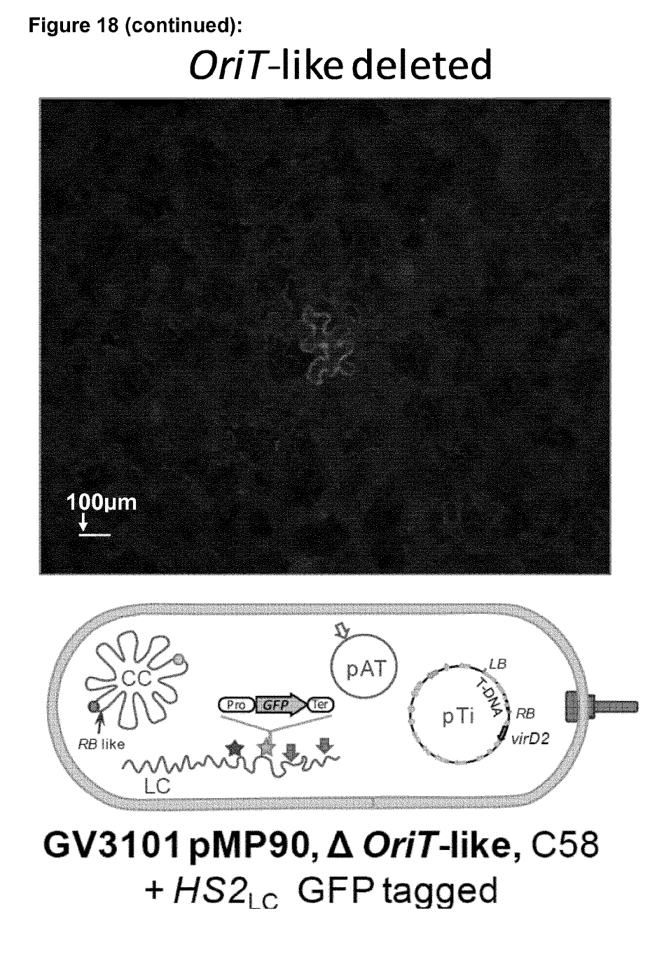

FIG. 18 shows that the deletion of OriT-like sequence from the linear chromosome hot spot 1 stopped also majority of chromosomal DNA transfer from linear chromosome hot spot 2 indicating that their transfer are linked and OriT-like sequence is responsible transfer of both regions to plants.

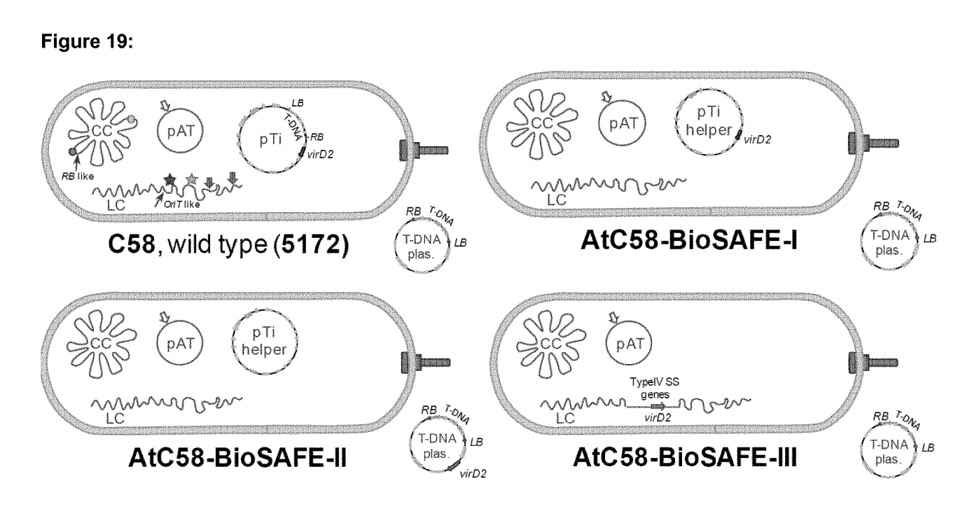

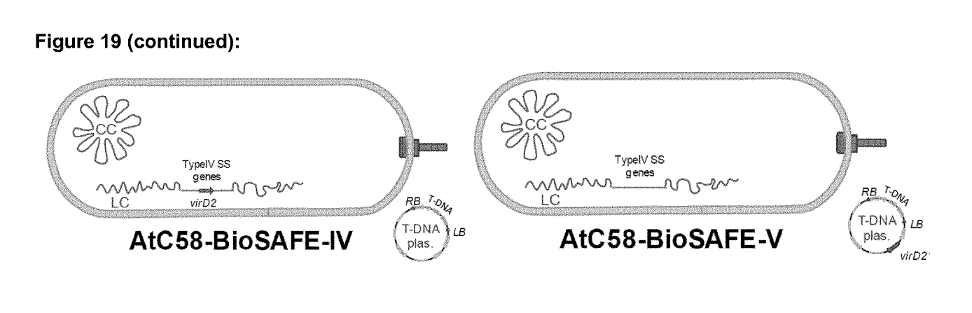

FIG. 19 shows schematic drawings of the genotypes of preferred BioSAFE Agrobacterium strains and associated vector systems according to the present invention.

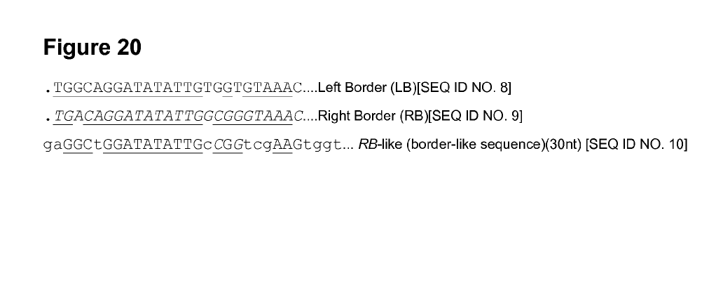

FIG. 20 shows an alignment identifying the left border, right border, and the border-like sequence according to the present invention based on alignments with RB or LB. LB, non italics; RB, italics; Nucleotides aligning neither LB nor RB are in small case, aligning sequences are in capital letters and are underlined.

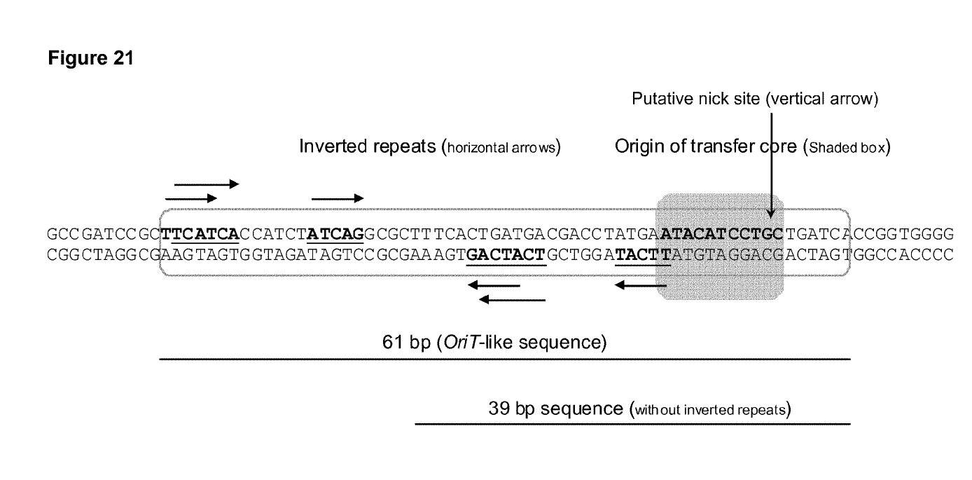

FIG. 21 shows that the predicted OriT-like sequence in the linear chromosome hot spot 1 is responsible for transferring this region from bacteria to plants, similar to FIG. 16A.

SEQ ID No. 1 to 3 show the sequences of IS426 copy I, II, and III, respectively. (see FIG. 6)

SEQ ID No. 4 to 6 show the amino acid sequences of ORFA, ORFB, and ORFAB, respectively. (see FIG. 6)

SEQ ID No. 7 shows the nucleotide sequence of the oriT region (61 bp). (see FIG. 17)

SEQ ID No. 8 and 9 show the right and left border sequences, respectively, and SEQ ID No. 10 shows the border-like sequence according to the invention. (see FIG. 20)

### EXAMPLES

In Europe and most of the world, Agrobacterium tumefaciens is classified under the risk group 1, therefore it can be used in research and development in all lowest security level (S1) laboratories. There are various Agrobacterium strains developed by researchers throughout the world. Recently, such strains are becoming commercially available (e.g. from Takara Bio, JP).

However, as the inventors have demonstrated in 2008, the available Agrobacterium strains have a hidden biosecurity risks. These bacteria appear to transfer very large fragments of its chromosomal DNA (AchrDNA) besides the DNA of interest which is typically cloned within the transferred region (T-DNA) whose limits are defined by 25 bp direct repeats which are termed “right and left border” (Ulker et al., 2008) (see FIG. 1).

Since many regions and plasmids are manipulated in the examples, FIG. 2 provides a simple diagram with acronyms and shapes are given for better comprehension of the work that has been done (see FIG. 2). Similarly, since many different strains of Agrobacterium with different genotypes are used, simple diagrams showing their characteristics are given in FIG. 3.

### 1. Mechanisms of AchrDNA Transfer: Transposon IS426

Promoter Trapping Resulted in Mostly IS426 Transposition into T-DNA Vector

In order to determine, how Agrobacterium chromosomal DNA fragments (AchrDNAs) other than T-DNAs are unintentionally transferred from the bacteria to plants, the inventors tested various possible mechanisms. Integration of T-DNA into bacterium's own chromosomes and a re-launch from the chromosomes together with some flanking AchrDNAs and their subsequent transfer to plants was one of the theories (Ulker et al., 2008). To test this theory, a trapping method which was expected to report insertion of T-DNA into Agrobacterium chromosomes was designed. The inventors called this method “insertional promoter trapping mediated kanamycin resistance” (IPTmKanR). The strategy relies on trapping promoters using a promoterless kanamycin resistance gene located at the right border of a T-DNA plasmid by growing bacteria on kanamycin containing LB plates (FIG. 4). Insertion of T-DNAs in various locations in bacterial chromosomes was expected as occasional insertions next to a promoter which could be sufficient to drive expression of the resistance gene and appearance of kanamycin resistant colonies.

The inventors obtained several kanamycin resistant Agrobacterium colonies. When the incubation time of plates at 28° C. was increased, the number of colonies resistant to kanamycin was also increased. Incubation of bacteria for five days on kanamycin selection plates resulted in between 40 to 80 colonies. Agrobacteria carrying no T-DNA plasmid or an unrelated plasmid without kanamycin resistance gene gave also 5-10 colonies, indicating that Agrobacterium has an alternative kanamycin resistance mechanism. The inventors picked more than 50 colonies appearing at different times on kanamycin plates. Interestingly, analysis of these colonies indicated that instead of trapping chromosomally integrated T-DNAs, mostly (˜61%) those cases were recovered, where an insertion sequence, IS426 copy from the Agrobacterium chromosomes was transposed upstream of the kanamycin resistance gene in the binary plasmid (FIG. 5). The rest of the resistant colonies were due to either rearrangements in plasmid or intrinsic resistance mechanisms present in the bacteria.

IS426 was first described in the literature in 1986, and was designated IS136 (Vanderleyden et al., 1986). Later, this name was changed to IS426. There were no other studies on this IS element. The study of Vanderleyden et al was short and did not contain detailed information. The authors reported that this IS element leads to a 9 bp duplication at the insertion site. However, later it was found that it leads to 5 bp duplications. A second publication appeared in 1999, and reported that the insertion of IS426 was responsible for disruption of tetracycline resistance in Agrobacterium (Luo and Farrand, 1999). Lately, other publications reporting the presence of IS426 in T-DNA plasmids were also published (Llop et al., 2009; Rawat et al., 2009), however none of these studies were directed at the characterization or removal of IS426 from the Agrobacterium genome. FIGS. 6 and 7 give some key features of this element relevant to biosafety.

### IS426 Copy Numbers and Transposition Mechanisms

Bioinformatics analysis showed the presence of two full-length and one partial copy of the IS426 in the sequenced A. tumefaciens C58 genome. The partial copy is located on the pTA plasmid, but both full-length copies are located on the linear chromosome. The full-length copies can be distinguished, because one of them has a three nucleotide (or one amino acid) deletion in the orfB region (FIG. 6). Bioinformatics analysis also suggested that IS426 has two putative, non-overlapping open reading frames (ORFs) (FIG. 6). The inventors furthermore detected a Chi sequence (AAAAAAA) between orfA and orfB. Such stretches of A's are shown to cause frame shifting during translation by ribosomes in other transposon coding sequences. Indeed, frame shifting in these A's of one nucleotide forward leads to orf-AB which is different from both orfA and orfB.

FIG. 6 shows the structure and putative coding sequences within IS426; there are two full length and one partial copy of the IS426 in the sequenced A. tumefaciens C58 genome. The full length IS426 I is 1319 bp and IS426 II is 1316 long (missing 3 nucleotides are highlighted by gray) and both are located on the linear chromosome. The partial copy is called IS426 III, and located on the pTA plasmid.

During PCR analysis with inverse primers specific for IS426, the inventors have also identified plasmid like episomal circles of IS426 in Agrobacterium cells (FIG. 7). These are possible transposition intermediates.

### Transposition Mechanism of IS426

To determine the transposition strategy of the IS426 element, the inventors developed a simple method. If the transposition is carried out by a cut and paste mechanism, the IS element should no longer be detected in the original sequenced location, however, if it is transposed as copy and paste mechanism, the IS element should retain its original location. Using analysis of the genomic DNA of IPTmKanR clones, where IS426 copies are transposed into this vector and integrated upstream of the nptII gene, the inventors found that the other copies of the IS426 are still in their original locations. This indicates that the mechanism of transposition functions not through cut and paste, but through copy and paste mechanism.

### Analysis of the IS426 Copy Number in the Most Frequently Used Agrobacterium Strains

High frequency of transposition into a plasmid upon antibiotic stress indicated that IS426 is an active transposon, and thus its copy number could have been different from the sequenced A. tumefaciens C58 strain. Therefore, the inventors performed DNA blot analysis with DNA isolated from A. tumefaciens C58 strain as well as several other important strains used in plant transformation. They combined DNA blot analysis data with inverse PCR and sequencing of the PCR fragments in order to determine the exact location of the IS426 copies. All three copies of the IS426 were found to be in the original location of the sequenced A. tumefaciens C58 strain (FIG. 8). This indicates that the copy number and the position of IS426 may be strictly controlled. A136 strain, a derivative of A. tumefaciens C58 lacking pTi plasmid carried also the same number of IS426 as the C58 strain, indicating that none of the IS426 copies is located on pTi plasmid.

Surprisingly, however, on DNA blot analysis the inventors detected an additional copy of IS426 in the engineered A. tumefaciens strain GV3101 pMP90. Rescuing the additional copy from the genomic DNA of this strain showed that this fourth copy of IS426 is located on the engineered Ti plasmid pMP90. The copy is inserted just upstream of virK gene whose function is still unknown (Hattori et al., 2001; Wilms et al., 2012). Analysis of LBA4404, an octopine type strain, by DNA blot analysis showed that this strain had either only one copy divergent from IS426, or a partial copy as indicated by a weak signal in the blot compared to other nopaline type strains.

### Deletion of IS426 Copies Using Homologous Recombination

After demonstration that IS426 can transpose into plasmids and may cause disruption of genes (virK, tetA, transgenes within T-DNA are some examples), activation of genes (nptII example), or unintentional transfer to plants by transposing into T-DNA regions of binary plant transformation vectors, it was desirable to completely remove this active IS426 element from the genome of the most frequently used Agrobacterium strains. This task was challenging, since there are two full and one partial copies of the IS426 as well as readily detected episomal IS426 circles. Furthermore, it was also possible that there would be a selective pressure for keeping these copies in their original location in the nopaline strains, and that removal of IS426 from these locations may cause adverse effects or may even be detrimental for the Agrobacterium strain.

In order to stepwise remove the active IS426 copies in the A136 model strain as used, the inventors generated homologous recombination vectors. These vectors, besides an antibiotic resistance gene, contained about 300 bp to 3000 bp flanking regions of the respective IS426 copies. Furthermore, the vectors lacked origin of replication regions for plasmid maintenance in Agrobacterium (suicide vector). Upon transformation into Agrobacterium and selection with appropriate antibiotics, it was expected that the antibiotic resistance gene in this suicide vectors recombines with the respective IS426 copy through homologous regions flanking the antibiotic resistance gene, and thus leads to a replacement of IS426 copy with the antibiotic resistance gene. Homologous recombination vectors with short homologies (300 to 400 bp) failed to delete the IS elements, however vectors having long homology stretches (1500 to 3000 bp) worked well and allowed the inventors to stepwise remove IS426-I and IS426-II. DNA blot analysis indicated that indeed these IS elements were indeed deleted from the A136 genome (FIG. 9). FIG. 9 shows the generation of IS426 deletion strains of Agrobacterium. Southern analysis to determine IS426 copy numbers in engineered, non-virulent, cured Agrobacterium strain A136 shows that both full copies are deleted.

### 2. Mechanisms of AchrDNA Transfer: OriT-Like and RB-Like Sequences

In order to determine the mechanisms of Agrobacterium chromosomal DNA (AchrDNA) transfer other than the IS426 element from Agrobacterium to plants the inventors developed a test system to rapidly determine the transfer of other AchrDNAs to plants. A planta expression cassette for green fluorescent protein (GFP) (containing 35S promoter and NOS terminator) was introduced into selected hot spots using homologous recombination with a suicide plasmid conferring spectinomycin or kanamycin resistance-genes (FIG. 10). The expected GFP tagging was confirmed by DNA blot (Southern) analyses in the engineered strains. Nicotiana benthamiana leaves were infiltrated with the engineered Agrobacterium strains, and GFP expression was determined three days post infiltration using a fluorescence microscope.

FIG. 10 shows that Agrobacterium chromosomal DNA hot spots could be labelled one by one by inserting a GFP expression cassette (active only in plants). The Agrobacterium strains generated were used in transiently transforming tobacco leaves. Expression of GFP in plant leaves was indicative of a T-DNA independent mechanism of AchrDNA transfer. This assay was then used in order to determine the biological and genetic conditions that are required to eliminate the transfer.

The tagging of the most frequently transferred hot spot on the linear chromosome of Agrobacterium (HS1_{LC}) with GFP in the GV3101 pMP90 strain and subsequent plant transformation assay showed that indeed such hot spots are transferred from bacterial chromosomes to plants (FIG. 11). In FIG. 11, the results of the homologous recombination mediated insertion of GFP tagging vector into the A. tumefaciens linear chromosome are shown. The recombination vector used is a suicide plasmid and cannot replicate in Agrobacterium. It contained the bacterial expression cassette for the spectinomycin resistance gene aadA1. A plant expression optimized GFP expression cassette flanked by the Agrobacterium sequences determined the position of the recombination mediated insertion into bacterial genome. The HS1_{LC} GFP-tagged Agrobacterium strains in the background of GV3101 pMP90 Agrobacterium cells were then used for the transient transformation of tobacco leaves to determine, whether these regions in the Agrobacterium genome are transferred to plants.

In addition and similar to HS1_{LC} tagging as above, also the second most frequently transferred hot spot on the linear chromosome of Agrobacterium (HS2_{LC}) was tagged with GFP in the GV3101 pMP90 strain. The subsequent plant transformation assay showed that this hot spot is transferred from bacterial chromosomes into plants (FIG. 12).

Then, the tagging unrelated non-hot spots in Agrobacterium chromosome with GFP gave no GFP expression in plants, which shows that DNA transfer is indeed site specific, as shown in FIG. 12, where tagging other hot spots and non-hot spots with GFP in GV3101 pMP90 Agrobacterium strain and plant transformation using transient assay is shown.

### AchrDNA Transfer is VirD2 and TypeIV SS Dependent

In addition to the T-DNA transfer system, Agrobacterium also contains many genes and secretion channels for conjugations of its plasmids. To determine how the DNA around hot spots are cleaved and transferred to plants, the inventors tagged the same regions in different Agrobacterium strains. Agrobacterium strain A136 was cured of the pTi plasmid, hence it has no TypeIV secretion system (SS) forming the injection channel, and VirD2 which is crucial in T-DNA transfer. On the other hand, the GV3101 pM600 ΔvirD2 strain contained the helper plasmid containing the TypeIV SS, but had a deletion of the virD2 gene. Thus, as shown in FIG. 13, a transfer of HS1_{LC} was blocked in both strains. This clearly indicates that like T-DNA, the processing of hot spot DNAs and their transfer into plants is both VirD2 and TypeIV SS dependent. In FIG. 13, the results of the tagging of HS1_{LC} with GFP in selected Agrobacterium strains show that the processing of chromosomal DNA is VirD2 and TypeIV SS dependent.

### VirD2 Cleavage Sites at or Around Hot Spots

Once it was determined that AchrDNA transfer is VirD2 dependent, the inventors searched for T-DNA right and left borders (RB and LB) or OriT sequences which were also shown to be cleavable by VirD2 (Pansegrau et al., 1993). Nevertheless, the analysis resulted in no perfect matches to these sequences, and many mismatches (as low as 65% match) had to be allowed. With such a low similarity, the inventors identified several hundred matches scattered throughout all chromosomes. The analysis was narrowed to around the hot spots, and tests with various sizes of fragments were performed in order to determine VirD2 cleavage sites. For this, PCR amplified fragments from selected regions on Agrobacterium genome were clone into pBasicS1-GFP plasmid (FIG. 14). pBasicS1-GFP, which can replicate in Agrobacterium, carries a GFP expression cassette that would report GFP expression in plants upon delivery. However, the plasmid had no T-DNA borders or origin of transfer sequences, therefore cannot transform plants with GFP. Fragments that were cloned into pBasicS1-GFP were transformed into GV3101 pMP90 Agrobacterium strain, and a transient plant transformation assay was carried out using N. benthamiana plants. As shown in FIG. 15, the inventors identified two key fragments (200 bp OriT-like in HS1_{LC} and 221 bp RB-like in HS2_{CC}) that contained VirD2 cleavage sites. FIG. 14 shows the map of the pBasicS1-GFP vector used in testing cis elements involved in chromosomal DNA transfer, and FIG. 15 shows the results of the search for DNA regions at or around the hot spots for a discovery of OriT-like and RB-like sequences responsible for AchrDNA transfer. PCR amplified fragments from selected regions on Agrobacterium genome were cloned into the pBasicS1-GFP plasmid (has no T-DNA borders or origin of transfer sequences, therefore cannot transform plants with GFP). The plasmids were then transformed into the GV3101 pMP90 Agrobacterium strain, and a transient plant transformation assay was carried out. Images were taken three days after infiltration with bacteria OD₆₀₀=0.25. Many other fragments were tested, but showed no GFP expression in plants.

In order to prove that the OriT-like sequence and the RB-like sequence as present in the 200 and 221 bp fragments are actually cleavage sites for VirD2, the inventors then generated shorter fragments that only contained the core sequence (61 bp OriT-like and 30 bp RB-like). FIG. 16A shows that—as expected—the predicted OriT-like sequence in the linear chromosome hot spot 1 is responsible for transferring this region from bacteria to plants. Origin of transfer sequences typically contain inverted repeats upstream of the core recognition sequence. Presence of these repeats is highly crucial for the functionality of the origin of transfer regions. As shown in FIG. 21, the inventors have also found such short inverted repeats and their deletion from the predicted origin of transfer (39 bp sequence) also eliminated its function and hence transfers of GFP to plants (FIGS. 16A and 21). Core OriT region which is contained within the 39 bp sequence has a limited similarity to the left and right borders. Since this fragment was not functional without the upstream inverted repeats, the activity of this sequence is not border but OriT. Images were taken six days after infiltration with bacteria OD₆₀₀=0.3. FIG. 16B shows that—as expected—the predicted RB-like sequence in the circular chromosome hot spot 1 is responsible for transferring this region from bacteria to plants. The 30 bp RB-like fragment was cloned into pBasicS1-GFP vector and tested in transient leaf transformation assays. Images were taken three days after infiltration with bacteria OD₆₀₀=0.3.

Deletion of OriT-Like from the Genome of Agrobacterium tumefacies Blocks the Vast Majority of Chromosomal DNA Transfer from HS1_{LC}

To further demonstrate that the elements as described above were responsible for chromosomal DNA transfer, the inventors first generated deletion mutants using homologous recombination for the OriT-like sequence on the linear chromosome. They used the HS1_{LC} GFP tagged GV3101 pMP90 Agrobacterium strain in order to knock out the OriT-like sequence (FIG. 17). Deletion of this sequence from the genome of the strain blocked the vast majority of chromosomal DNA transfer into this locus (FIG. 18). Some positive cells were still found, indicating that there may be at least one more sequence involved in a weak DNA transfer in or around this hot spot. FIG. 17 shows the results of the homologous recombination mediated deletion of OriT-like sequence from the A. tumefaciens linear chromosome; and the strategy used in the deletion of the OriT-like sequences is depicted. (A) The recombination vector is a suicide plasmid and cannot replicate in Agrobacterium. It contains the bacterial expression cassette for the kanamycin resistance gene nptII, flanked by the Agrobacterium sequences determining the position of recombination mediated deletion of sequences from bacterial genome by the introduction of the nptII expression cassette. (B) Transformation of the recombination vector into HS1_{LC} GFP-tagged GV3101 pMP90 Agrobacterium cells and selection of bacteria by kanamycin resulted in double recombination mediated replacement of nptII with the OriT-like sequence on the linear chromosome. FIG. 18 shows the results of the deletion of the OriT-like sequence from the linear chromosome hot spot 1, which stopped the vast majority of chromosomal DNA transfer from this hot spot. The homologous recombination-mediated deletion of OriT-like sequence was carried out in the GV3101 pMP90 strain, where hot spot 1 was tagged with the GFP expression cassette. Only very few positive plant cells were obtained comprising a deleted OriT-like sequence, suggesting that this sequence is the main source of chromosomal DNA transfer. Images were taken three days after infiltration with bacteria OD₆₀₀=0.3.

HS1_{LC} and HS2_{LC} are Linked, and the Deletion of OriT-Like from the Genome of Agrobacterium Tumefacies Also Blocks the Vast Majority of the Chromosomal DNA Transfer from HS2_{LC}

The second most frequently transferred hot spot on Agrobacterium chromosomes is HS2_{LC}, and this hot spot is located about 30 Kb downstream from HS1_{LC}, indicating that they may be linked. In order to determine, whether the transfer of these hot spots is linked, and whether the DNA transfer process is initiated at the OriT-like sequence, the inventors deleted the OriT-like sequence from HS2_{LC} GFP tagged GV3101 pMP90 Agrobacterium strain. Like in the case of HS1_{LC}, the transfer of HS2_{LC} into plants cells was mostly abolished, indicating that these hot spots are linked and DNA transfers are initiated at OriT-like sequence at HS1_{LC} (FIG. 18). Furthermore, the inventors identified only very few positive cells, indicating again that there may be least one more weak sequence involved in DNA transfer in or around these hot spots. FIG. 18 shows the results of the deletion of OriT-like sequence from the linear chromosome hot spot 1, which stopped also the vast majority of chromosomal DNA transfer from linear chromosome hot spot 2; indicating that their transfers are linked, and that the OriT-like sequence is responsible transfer of both regions to plants. The homologous recombination mediated deletion of OriT-like sequence was carried out in GV3101 pMP90 strain, where hot spot 2 was tagged with GFP expression cassette. Images were taken three days after infiltration with bacteria OD₆₀₀=0.3.

### Combination of the Deletions in the Genome of Agrobacterium Tumefacies

A strain of Agrobacterium is constructed that combines the deletions of the OriT-like, RB-like and IS426 copies as described above. This Agrobacterium strain shows only extremely low AchrDNA transfer to plants.

As a particular example, the AtC58-BioSAFE bacterium has the genotype of a deletion of the 61 bp OriT-like element in the HS1_{LC} region on the linear chromosome, a deletion of the 30 bp RB-like sequence in the HS1_{CC} region on the circular chromosome, and deletions of the two full length insertion sequences, IS426 copy I and IS426 copy II from the linear chromosome.

Furthermore, the strain will optionally contain the chromosomally integrated minimal Type IV secretion system (TypeIV SS). This will simplify plant transformation because there will be no more need for a binary system and helper plasmids. There are two alternatives, TypeIV SS containing virD2 or not containing virD2. Transferring the core components of the TypeIV secretion system (TypeIV SS) from pTi plasmid into Agrobacterium linear chromosomes simplifies the so called binary (dual) vector system in plant transformation into a unitary (single component) system. In the binary system, the components of the DNA transfer machinery (tumor inducing plasmid, pTi plasmid) were divided into two plasmids (two components). The TypeIV SS (component one, also called the helper plasmid) forms the bacterial injection system as well as contains the key genes involved in processing and transferring T-DNA into plants. In the original pTi plasmid, there were genes causing tumor formation in plants within the T-DNA region. Therefore, this region is completely deleted from the helper plasmids. However, in order to transform plants with a desired DNA, a T-DNA vector where the 25 bp borders are present (but no longer the tumor causing genes) is necessary. Therefore, various T-DNA vectors (component two) were generated to aid researchers for cloning gene of interests within the T-DNA for plant transformation.

FIG. 19 shows the desired BioSAFE Agrobacterium strains and associated vector systems. The AtC58-BioSAFE-I and II strains are in classical binary system except for the deleted sequences as described above. The virD2 coding sequence from the helper plasmid is deleted to generate the AtC58-BioSAFE-II. The deleted virD2 will be supplied again with the complementing plant transformation vector. The AtC58-BioSAFE-III, IV and V strains are in the unitary system as the minimal TypeIV SS genes necessary for channel formation and DNA/protein delivery to eukaryotic cells are inserted into the liner chromosome of the Agrobacterium. The AtC58-BioSAFE-III contains the pAT plasmid but the AtC58-BioSAFE-IV and V is devoid of it. The difference between AtC58-BioSAFE-IV and V is that virD2 is absent in the AtC58-BioSAFE-V genome, but will be supplied with the complementing plasmid vector.

### REFERENCES AS CITED

Ülker, B., Li, Y., Rosso, M. G., Logemann, E., Somssich, I. E., and Weisshaar, B. (2008). T-DNA-mediated transfer of Agrobacterium tumefaciens chromosomal DNA into plants. Nat Biotechnol 26, 1015-1017.
Berson, T., Stirnberg, A. and Ülker, B. (2014). Characterization and elimination of IS426, an active insertion sequence of Agrobacterium tumefaciens. (in press)
Gelvin, S. B. (2008). Agrobacterium-mediated DNA transfer, and then some. Nat Biotechnol 26, 998-1000.
Grove, J. I., Alandiyjany, M. N., and Delahay, R. M. (2013). Site-specific Relaxase Activity of a VirD2-like Protein Encoded within the tfs4 Genomic Island of Helicobacter pylori. J Biol Chem 288: 26385-26396.
Hattori, Y., Iwata, K., Suzuki, K., Uraji, M., Ohta, N., Katoh, A., and Yoshida, K. (2001). Sequence characterization of the vir region of a nopaline type Ti plasmid, pTi-SAKURA. Genes Genet Syst 76: 121-130.
Llop, P., Murillo, J., Lastra, B., and Lopez, M. M. (2009). Recovery of nonpathogenic mutant bacteria from tumors caused by several Agrobacterium tumefaciens strains: a frequent event? Appl Environ Microbiol 75: 6504-6514.
Luo, Z. Q., and Farrand, S. K. (1999). Cloning and characterization of a tetracycline resistance determinant present in Agrobacterium tumefaciens C58. J Bacteriol 181: 618-626.
Pansegrau, W., Schoumacher, F., Hohn, B., and Lanka, E. (1993). Site-specific cleavage and joining of single-stranded DNA by VirD2 protein of Agrobacterium tumefaciens Ti plasmids: analogy to bacterial conjugation. Proc Natl Acad Sci USA 90: 11538-11542.
Rawat, P., Kumar, S., Pental, D., and Burma, P. K. (2009). Inactivation of a transgene due to transposition of insertion sequence (IS136) of Agrobacterium tumefaciens. J Biosci 34: 199-202.
Vanderleyden, J., Desair, J., De Meirsman, C., Michiels, K., Van Gool, A. P., Chilton, M. D., and Jen, G. C. (1986). Nucleotide sequence of an insertion sequence (IS) element identified in the T-DNA region of a spontaneous variant of the Ti-plasmid pTiT37. Nucleic Acids Res 14: 6699-6709.

Wilms, I., Overloper, A., Nowrousian, M., Sharma, C. M., and Narberhaus, F. (2012). Deep sequencing uncovers numerous small RNAs on all four replicons of the plant pathogen Agrobacterium tumefaciens. RNA Biol 9: 446-457.

1. An agrobacterium tumefaciens strain, comprising at least one deletion in a sequence selected from the group consisting of IS426 copy I, IS426 copy II, the OriT-like sequence, and border-like sequences.

2. The Agrobacterium tumefaciens strain according to claim 1, wherein said strain comprises a deletion in at least two of said sequences.

3. The Agrobacterium tumefaciens strain according to claim 1, wherein the deletion of said sequence is a partial deletion of the sequence.

4. The Agrobacterium tumefaciens strain according to claim 1, wherein said strain comprises one or more nucleotide changes in at least one of said sequences.

5. The Agrobacterium tumefaciens strain according to claim 1, wherein the OriT-like sequence is located in the HS1_{LC} region and the RB-like sequence is located in the HS1_{CC} region.

6. The Agrobacterium tumefaciens strain according to claim 1, further comprising either a helper plasmid containing a TypeIV secretion system or a recombinant chromosomally integrated minimal TypeIV secretion system (TypeIV SS).

7. A method for producing an Agrobacterium tumefaciens strain according to claim 1, comprising the step of introducing at least one deletion and/or inactivating/mutation in a sequence selected from the group consisting of IS426 copy I, IS426 copy II, OriT-like sequence, and RB-like sequence in said strain.

8. A method for producing an Agrobacterium tumefaciens strain, comprising introducing a recombinant chromosomally: integrated minimal TypeIV secretion system (TypeIV SS) into an Agrobacterium tumefaciens strain according to claim 1.

9. A method for transforming a cell selected from the group consisting of a plant, yeast, fungal, and human cell with a recombinant nucleic acid, comprising contacting said cell with an Agrobacterium tumefaciens strain according to claim 1 carrying said recombinant nucleic acid to be transformed.

10. The Agrobacterium tumefaciens strain according to claim 1, wherein said strain comprises a deletion in at least three of said sequences.

11. The Agrobacterium tumefaciens strain according to claim 1, wherein said strain comprises a deletion in all four of said sequences.

12. The Agrobacterium tumefaciens strain according to claim 1, wherein the deletion of said sequence is 30 bp in the RB-like sequence, and/or 61 bp in the OriT-like sequence.

13. The Agrobacterium tumefaciens strain according to claim 1, wherein the deletion of said sequence is full deletion of the sequence.

14. The Agrobacterium tumefaciens strain according to claim 6, further comprising virD2.

15. The method for producing an Agrobacterium tumefaciens strain, according to claim 8, further comprising introducing a recombinant chromosomally-integrated virD2, into the Agrobacterium tumefaciens strain.

16. The method, according to claim 7, wherein said method comprises introducing a deletion in at least two of said sequences.

17. The method, according to claim 7, wherein the deletion of said sequence is a partial deletion of the sequence.

18. The method, according to claim 7, wherein the deletion of said sequence is 30 bp in the RB-like sequence, and/or 61 bp in the OriT-like sequence.

19. The method, according to claim 7, wherein the OriT-like sequence is located in the HS1_{LC} region and the RB-like sequence is located in the HS1_{CC} region.

20. The method, according to claim 7, wherein said strain further comprises either a helper plasmid containing a TypeIV secretion system or a recombinant chromosomally integrated minimal TypeIV secretion system (TypeIV SS).


SEQUENCE LISTING (10 sequences)

SEQ ID NO: 1 - DNA, 1319 bp, Agrobacterium tumefaciens
tgaactgccccccatttcgaccggacagtcggcataagcagaaaggctcaagcacaggcttgaggacaggcttatgtctaacgactatcgacacgttgaattgctgacgggtgatgttcgccgcaggcggtggacaaccgagcaaaagctgacaatcattgagcagagttttgaacccggcgagacggtatcttcgaccgctcgccgtcatggcgtggcgcccaatttgctttatcggtggcgcaggctcttgagcgagggaggtgctgcagccgtggattctgacgagccggttgtcgggaattcggaagtgaagaaactggaggatcgcgtccgggagttggagcgcatgctcggtcgcaagacgatggaggtcgaaatcctccgcgaagccctttccaaagcggactcaaaaaaacggatatcgcggccgatcttgttgccgaaggacggttcgcgatgaaggccgtcgcagacacgctgggcgtctcccgttccaacctcatcgagcggctgaaaggcagatcaaagccgcgtgggccatacaacaaggccgaggatgcagagcttctgcccgccatccgcaggctggtggatcaaaggccaacctatggctatcggcggatcgccgcgctcctcaatcgcgaaaggcgagccgccgatcagcctgtcgtcaacgccaaacgggtccatcgcatcatgggtaaccacgccatgctactggagaagcacacagccgttcgcaagggccgcctccacgatggcaaggtcatggtcatgcgctccaacctgcgctggtgctcggacggcctggagttcgcctgctggaatggcgaggtcattcgtctcgccttcatcatcgacgccttcgaccgcgagatcatcgcctggacggccgttgccaatgcaggcatttccggctcagacgtgcgcgacatgatgttggaggcggtcgagaaacgcttccatgcaacccgagccccgcatgctatcgagcatctctctgacaatggctcggcttataccgcgcgggacacgaggctgtttgcgcaagcactcaatctcacgccctgcttcacgccggtcgccagcccgcagtcgaacggcatgtcggaagccttcgtcaaaacgttgaagcgggactatattcggatatcagctctaccggacgcccaaacagcgctccggctcatcgacggatggatcgaggactacaacgaaatccatccccattccgcgctcaagatggcttcccctcggcagttcatcagggctaaatcaatctagccgacttgtccggtgaaatggggtgcactcca

SEQ ID NO: 2 - DNA, 1316 bp, Agrobacterium tumefaciens
tgaactgccccccatttcgaccggacagtcggcataagcagaaaggctcaagcacaggcttgaggacaggcttatgtctaacgactatcgacacgttgaattgctgacgggtgatgttcgccgcaggcggtggacaaccgagcaaaagctgacaatcattgagcagagttttgaacccggcgagacggtatcttcgaccgctcgccgtcatggcgtggcgcccaatttgctttatcggtggcgcaggctcttgagcgagggaggtgctgcagccgtggattctgacgagccggttgtcgggaattcggaagtgaagaaactggaggatcgcgtccgggagttggagcgcatgctcggtcgcaagacgatggaggtcgaaatcctccgcgaagccctttccaaagcggactcaaaaaaacggatatcgcggccgatcttgttgccgaaggacggttcgcgatgaaggccgtcgcagacacgctgggcgtctcccgttccaacctcatcgagcggctgaaaggcagatcaaagccgcgtgggccatacaacaaggccgaggatgcagagcttctgcccgccatccgcaggctggtggatcaaaggccaacctatggctatcggcggatcgccgcgctcctcaatcgcgaaaggcgagccgccgatcagcctgtcgtcaacgccaaacgggtccatcgcatcatgggtaaccacgccatgctactggagcacacagccgttcgcaagggccgcctccacgatggcaaggtcatggtcatgcgctccaacctgcgctggtgctcggacggcctggagttcgcctgctggaatggcgaggtcattcgtctcgccttcatcatcgacgccttcgaccgcgagatcatcgcctggacggccgttgccaatgcaggcatttccggctcagacgtgcgcgacatgatgttggaggcggtcgagaaacgcttccatgcaacccgagccccgcatgctatcgagcatctctctgacaatggctcggcttataccgcgcgggacacgaggctgtttgcgcaagcactcaatctcacgccctgcttcacgccggtcgccagcccgcagtcgaacggcatgtcggaagccttcgtcaaaacgttgaagcgggactatattcggatatcagctctaccggacgcccaaacagcgctccggctcatcgacggatggatcgaggactacaacgaaatccatccccattccgcgctcaagatggcttcccctcggcagttcatcagggctaaatcaatctagccgacttgtccggtgaaatggggtgcactcca

SEQ ID NO: 3 - DNA, 955 bp, Agrobacterium tumefaciens
gccccccatttcgaccggacagtctgcataagcagaaaggctcaagcacaggcttggttataggcttatgtctaacgagtatcgacacgttgaattgctgacgggtgatgttcgccccaggcggtggacaaccgagcaaaagctgacaatcattgagcagagtttcttacccggcgagacggtatcttcgaccgctcgccgtcatggcgtcgcgcccaatttgctttatcggtggcgcaggctcttgagcgagggaggtgctgcagccgtggattctgacgagccggttgtcgggaattcggaagtgaagaaactggaggatcgcgtccgggagttggagcgcatgctcagtcgcaagacgatggaggtcgaatcctccgcgaagcccttaccaaaaaaacggatatcgcggccgatcttgttgccgaaggacggttcgcgatgaagaccgtcgcagacacgctgggcgtctcccgatccaacctcatcgagcggctgaaaggcaaatcaaagccgcgtgggccatacaacaaggtcgaggatgcagagattttgcccatcatccgcaggctggtggatcaaatgccaacctatggctatcggcggatcgccgcgctcctcaatcgcgaagggcgagccgccgataagcctgtcgtcaacgctaaacgggttcatcgcattatgggcaaccacgcgatgctgctcgagaagcacacagccgttcgcaagggccgcctccacgacggcaaggtgatggtcgtgcgctcgaacttgcgctggtgctcggatgggctggagttcacttgctggaacggcgaggtcatccgtctcgccttcatcatcgacgctttcgactgggatatcatcgcctggacggcggccgccaacgcgggcatctccggctctgacgtgcgcgacatgatgctggaggcggtcgaaaagcggttcgcagcaa

SEQ ID NO: 4 - protein, 129 aa, Agrobacterium tumefaciens
MSNDYRHVELLTGDVRRRRWTTEQKLTIIEQSFEPGETVSSTARRHGVAPNLLYRWRRLLSEGGAAAVDSDEPVVGNSEVKKLEDRVRELERMLGRKTMEVEILREALSKADSKKRISRPILLPKDGSR

SEQ ID NO: 5 - protein, 275 aa, Agrobacterium tumefaciens
MKAVADTLGVSRSNLIERLKGRSKPRGPYNKAEDAELLPAIRRLVDQRPTYGYRRIAALLNRERRAADQPVVNAKRVHRIMGNHAMLLEKHTAVRKGRLHDGKVMVMRSNLRWCSDGLEFACWNGEVIRLAFIIDAFDREIIAWTAVANAGISGSDVRDMMLEAVEKRFHATRAPHAIEHLSDNGSAYTARDTRLFAQALNLTPCFTPVASPQSNGMSEAFVKTLKRDYIRISALPDAQTALRLIDGWIEDYNEIHPHSALKMASPRQFIRAKSI

SEQ ID NO: 6 - protein, 404 aa, Agrobacterium tumefaciens
MSNDYRHVELLTGDVRRRRWTTEQKLTIIEQSFEPGETVSSTARRHGVAPNLLYRWRRLLSEGGAAAVDSDEPVVGNSEVKKLEDRVRELERMLGRKTMEVEILREALSKADSKKTDIAADLVAEGRFAMKAVADTLGVSRSNLIERLKGRSKPRGPYNKAEDAELLPAIRRLVDQRPTYGYRRIAALLNRERRAADQPVVNAKRVHRIMGNHAMLLEKHTAVRKGRLHDGKVMVMRSNLRWCSDGLEFACWNGEVIRLAFIIDAFDREIIAWTAVANAGISGSDVRDMMLEAVEKRFHATRAPHAIEHLSDNGSAYTARDTRLFAQALNLTPCFTPVASPQSNGMSEAFVKTLKRDYIRISALPDAQTALRLIDGWIEDYNEIHPHSALKMASPRQFIRAKSI

SEQ ID NO: 7 - DNA, 61 bp, Agrobacterium tumefaciens
ttcatcaccatctatcaggcgctttcactgatgacgacctatgaatacatcctgctgatca

SEQ ID NO: 8 - DNA, 25 bp, Agrobacterium tumefaciens
tggcaggatatattgtggtgtaaac

SEQ ID NO: 9 - DNA, 25 bp, Agrobacterium tumefaciens
tgacaggatatattggcgggtaaac

SEQ ID NO: 10 - DNA, 30 bp, Agrobacterium tumefaciens
gaggctggatatattgccggtcgaagtggt